(19) United States
(12) Patent Application Publication
(10) Pub. No.: US 2019/0072486 A1
(43) Pub. Date: Mar. 7, 2019

(54) TRAPEZOIDAL FLAT PLATE TYPE SURFACE PLASMON RESONANCE SENSING PLATFORM

(71) Applicant: MING CHUAN UNIVERSITY, Taipei (TW)

(72) Inventors: Yu-Cheng LIN, Taipei (TW); Bo-Yu LIN, Taipei (TW)

(21) Appl. No.: 15/693,590

(22) Filed: Sep. 1, 2017

(51) Int. Cl. G01N 21/552; G01N 33/483

(52) U.S. Cl. CPC G01N 21/554; G01N 33/483

(57) ABSTRACT

A trapezoidal flat plate type surface plasmon resonance sensing platform is applied to a sensed object, a light-emitting apparatus and a light-analyzing apparatus. The trapezoidal flat plate type surface plasmon resonance sensing platform includes a trapezoidal flat plate and a metal layer. The metal layer is arranged on the trapezoidal flat plate. The sensed object is arranged on the metal layer. The light-emitting apparatus emits a testing light entering the trapezoidal flat plate, so that the testing light is refracted and reflected in the trapezoidal flat plate to generate a plurality of total reflections and leaves from the trapezoidal flat plate to generate an external refracted light. The light-analyzing apparatus receives the external refracted light to analyze the external refracted light. Moreover, the total reflections are used to excite a plurality of free electrons of the metal layer to generate a surface plasmon resonance phenomenon.

### BACKGROUND OF THE INVENTION

### Field of the Invention

The present invention relates to a surface plasmon resonance sensing platform, and especially relates to a trapezoidal flat plate type surface plasmon resonance sensing platform.

### Description of the Related Art

The application range of the related art surface plasmon resonance sensing platform is very wide, for examples the drug research, medical diagnosis, environmental monitoring and food safety, so that the related art surface plasmon resonance sensing platform is very important. Currently, the technology of the related art surface plasmon resonance sensing platform is quite mature.

However, numerous of the related art surface plasmon resonance sensing platforms utilize the prism designs, so that the related art surface plasmon resonance sensing platforms comprise the disadvantages of difficultly processing, high cost, unable to largely produce and too complicated structure. In another word, the related art surface plasmon resonance sensing platforms at least comprise the following disadvantages:

1. The incident angle of the testing light entering the prism sensing platform has to be adjusted accurately, so that the main energy of the testing light is bale to touch the test area.

2. After the testing light performs a total reflection in the prism sensing platform, the testing light leaves from the prism sensing platform, so that the signal strength is not strong.

### SUMMARY OF THE INVENTION

In order to solve the above-mentioned problems, an object of the present invention is to provide a trapezoidal flat plate type surface plasmon resonance sensing platform

In order to achieve the object of the present invention mentioned above, the trapezoidal flat plate type surface plasmon resonance sensing platform of the present invention is applied to a sensed object, a light-emitting apparatus and a light-analyzing apparatus. The trapezoidal flat plate type surface plasmon resonance sensing platform comprises a trapezoidal flat plate and a metal layer. The metal layer is arranged on the trapezoidal flat plate. The sensed object is arranged on the metal layer. The light-emitting apparatus emits a testing light entering the trapezoidal flat plate, so that the testing light is refracted and reflected in the trapezoidal flat plate to generate a plurality of total reflections and leaves from the trapezoidal flat plate to generate an external refracted light. The light-analyzing apparatus receives the external refracted light to analyze the external refracted light. Moreover, the total reflections are used to excite a plurality of free electrons of the metal layer to generate a surface plasmon resonance phenomenon.

Moreover, in an embodiment of the above-mentioned trapezoidal flat plate type surface plasmon resonance sensing platform of the present invention, the trapezoidal flat plate comprises an upper base and a lower base. A length of the upper base of the trapezoidal flat plate is longer than a length of the lower base of the trapezoidal flat plate. The upper base of the trapezoidal flat plate is parallel to the lower base of the trapezoidal flat plate. The metal layer is arranged on the upper base of the trapezoidal flat plate. The testing light is refracted in the trapezoidal flat plate and is reflected on the lower base of the trapezoidal flat plate to generate the total reflections on the upper base of the trapezoidal flat plate.

Moreover, in an embodiment of the above-mentioned trapezoidal flat plate type surface plasmon resonance sensing platform of the present invention, the trapezoidal flat plate comprises a light-entering broadside and a light-leaving broadside. A length of the light-entering broadside of the trapezoidal flat plate is equal to a length of the light-leaving broadside of the trapezoidal flat plate. The light-entering broadside of the trapezoidal flat plate is not parallel to the light-leaving broadside of the trapezoidal flat plate. The light-emitting apparatus emits the testing light entering the trapezoidal flat plate through the light-entering broadside of the trapezoidal flat plate and leaves from the trapezoidal flat plate through the light-leaving broadside of the trapezoidal flat plate to generate the external refracted light.

Moreover, in an embodiment of the above-mentioned trapezoidal flat plate type surface plasmon resonance sensing platform of the present invention, an incident angle of the testing light entering the trapezoidal flat plate is, for example but not limited to, eight degrees.

Moreover, in an embodiment of the above-mentioned trapezoidal flat plate type surface plasmon resonance sensing platform of the present invention, the trapezoidal flat plate and the metal layer are integrated as a removable module.

Moreover, in an embodiment of the above-mentioned trapezoidal flat plate type surface plasmon resonance sensing platform of the present invention, the metal layer is sputtered on the trapezoidal flat plate.

Moreover, in an embodiment of the above-mentioned trapezoidal flat plate type surface plasmon resonance sensing platform of the present invention, the metal layer is divided into a sensing area array to sense a plurality of the sensed objects.

Moreover, in an embodiment of the above-mentioned trapezoidal flat plate type surface plasmon resonance sensing platform of the present invention, the trapezoidal flat plate is, for example but not limited to, a trapezoidal glass flat plate.

Moreover, in an embodiment of the above-mentioned trapezoidal flat plate type surface plasmon resonance sensing platform of the present invention, the light-analyzing apparatus is, for example but not limited to, a frequency spectrum analyzer.

Moreover, in an embodiment of the above-mentioned trapezoidal flat plate type surface plasmon resonance sensing platform of the present invention, the sensed object is, for example but not limited to, a saliva sample, a blood sample, a hair sample, an egesta sample or a cell sample.

The advantage of the present invention is to arrange the surface plasmon resonance sensing platform easily and increase the signal strength.

Please refer to the detailed descriptions and figures of the present invention mentioned below for further understanding the technology, method and effect of the present invention. The figures are only for references and descriptions, and the present invention is not limited by the figures.

### BRIEF DESCRIPTION OF DRAWING

FIG. 1 shows the first embodiment of the trapezoidal flat plate type surface plasmon resonance sensing platform of the present invention.

FIG. 2 shows the second embodiment of the trapezoidal flat plate type surface plasmon resonance sensing platform of the present invention.

FIG. 3 shows the third embodiment of the trapezoidal flat plate type surface plasmon resonance sensing platform of the present invention.

### DETAILED DESCRIPTION OF THE INVENTION

Please refer to following detailed description and figures for the technical content of the present invention:

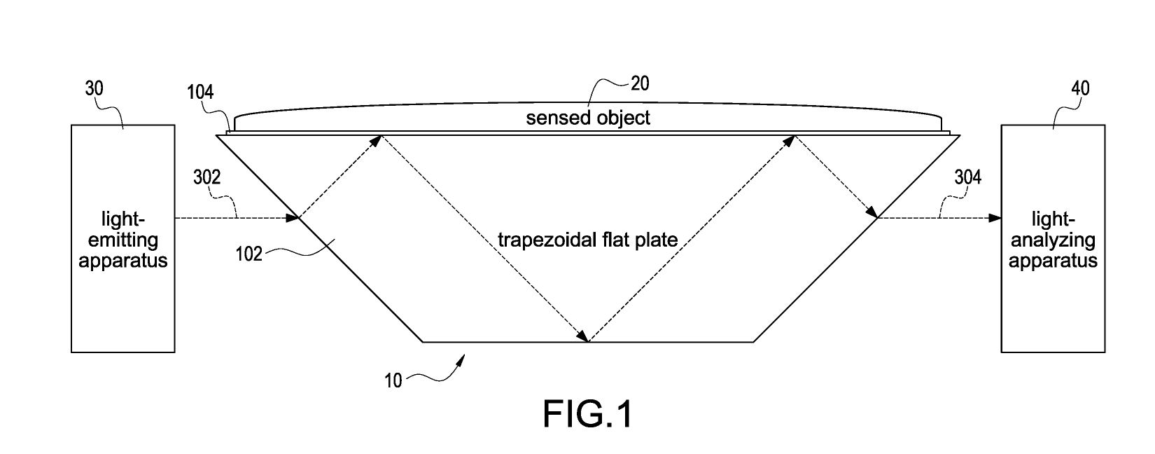

FIG. 1 shows the first embodiment of the trapezoidal flat plate type surface plasmon resonance sensing platform of the present invention. A trapezoidal flat plate type surface plasmon resonance sensing platform 10 of the present invention is applied to a sensed object 20, a light-emitting apparatus 30 and a light-analyzing apparatus 40. The trapezoidal flat plate type surface plasmon resonance sensing platform 10 comprises a trapezoidal flat plate 102 and a metal layer 104. The metal layer 104 is arranged on the trapezoidal flat plate 102. The sensed object 20 is arranged on the metal layer 104.

The light-emitting apparatus 30 emits a testing light 302 entering the trapezoidal flat plate 102, so that the testing light 302 is refracted and reflected in the trapezoidal flat plate 102 to generate a plurality of total reflections and leaves from the trapezoidal flat plate 102 to generate an external refracted light 304. The light-analyzing apparatus 40 receives the external refracted light 304 to analyze the external refracted light 304. The total reflections are used to excite a plurality of free electrons of the metal layer 104 to generate a surface plasmon resonance phenomenon.

In an embodiment of the present invention, an incident angle of the testing light 302 entering the trapezoidal flat plate 102 is, for example but not limited to, eight degrees. The trapezoidal flat plate 102 and the metal layer 104 are integrated as a removable module (to benefit the metal sputtering to increase the convenience of the structure). The metal layer 104 is sputtered on the trapezoidal flat plate 102. The metal layer 104 is divided into a sensing area array to sense a plurality of the sensed objects 20. The trapezoidal flat plate 102 is, for example but not limited to, a trapezoidal glass flat plate. The light-analyzing apparatus 40 is, for example but not limited to, a frequency spectrum analyzer. The sensed object 20 is, for example but not limited to, a saliva sample, a blood sample, a hair sample, an egesta sample or a cell sample.

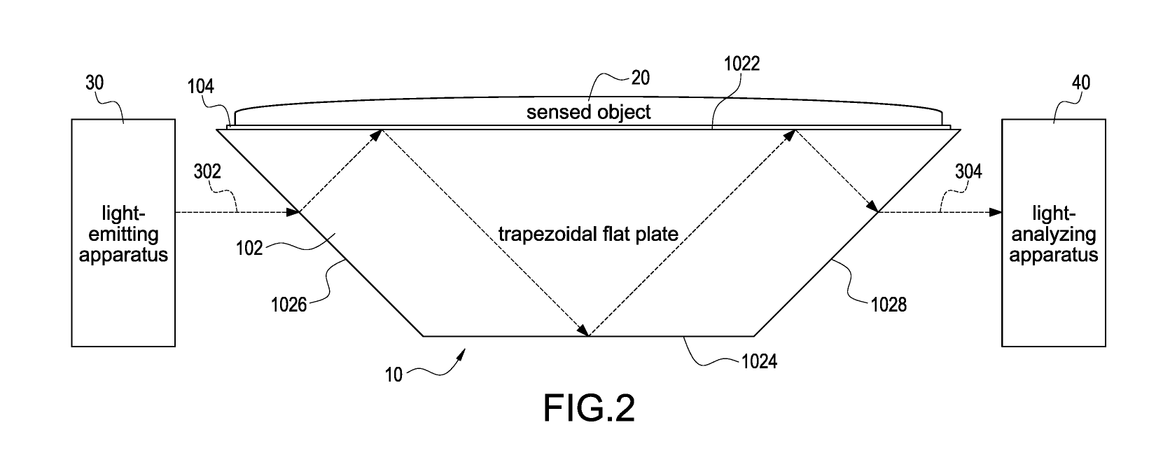

FIG. 2 shows the second embodiment of the trapezoidal flat plate type surface plasmon resonance sensing platform of the present invention. A trapezoidal flat plate type surface plasmon resonance sensing platform 10 of the present invention is applied to a sensed object 20, a light-emitting apparatus 30 and a light-analyzing apparatus 40. The trapezoidal flat plate type surface plasmon resonance sensing platform 10 comprises a trapezoidal flat plate 102 and a metal layer 104. The metal layer 104 is arranged on the trapezoidal flat plate 102. The sensed object 20 is arranged on the metal layer 104. The trapezoidal flat plate 102 comprises an upper base 1022, a lower base 1024, a light-entering broadside 1026 and a light-leaving broadside 1028.

A length of the upper base 1022 of the trapezoidal flat plate 102 is longer than a length of the lower base 1024 of the trapezoidal flat plate 102. The upper base 1022 of the trapezoidal flat plate 102 is parallel to the lower base 1024 of the trapezoidal flat plate 102. The metal layer 104 is arranged on the upper base 1022 of the trapezoidal flat plate 102. The testing light 302 is refracted in the trapezoidal flat plate 102 and is reflected on the lower base 1024 of the trapezoidal flat plate 102 to generate the total reflections on the upper base 1022 of the trapezoidal flat plate 102.

A length of the light-entering broadside 1026 of the trapezoidal flat plate 102 is equal to a length of the light-leaving broadside 1028 of the trapezoidal flat plate 102. The light-entering broadside 1026 of the trapezoidal flat plate 102 is not parallel to the light-leaving broadside 1028 of the trapezoidal flat plate 102. The light-emitting apparatus 30 emits the testing light 302 entering the trapezoidal flat plate 102 through the light-entering broadside 1026 of the trapezoidal flat plate 102 and leaves from the trapezoidal flat plate 102 through the light-leaving broadside 1028 of the trapezoidal flat plate 102 to generate the external refracted light 304. The descriptions of the elements shown in FIG. 2 which are the same as the elements shown in FIG. 1 are not repeated here for brevity.

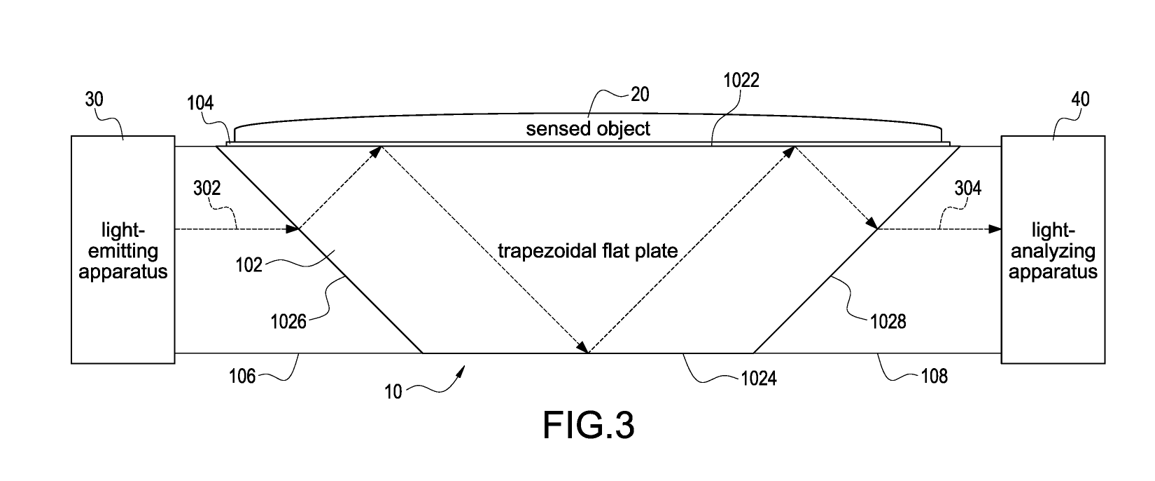

FIG. 3 shows the third embodiment of the trapezoidal flat plate type surface plasmon resonance sensing platform of the present invention. A trapezoidal flat plate type surface plasmon resonance sensing platform 10 of the present invention is applied to a sensed object 20, a light-emitting apparatus 30 and a light-analyzing apparatus 40. The trapezoidal flat plate type surface plasmon resonance sensing platform 10 comprises a trapezoidal flat plate 102 and a metal layer 104. The metal layer 104 is arranged on the trapezoidal flat plate 102. The sensed object 20 is arranged on the metal layer 104. The trapezoidal flat plate 102 comprises an upper base 1022, a lower base 1024, a light-entering broadside 1026 and a light-leaving broadside 1028.

The trapezoidal flat plate type surface plasmon resonance sensing platform 10 further comprises a light-emitting side optical fiber 106 and a light-receiving side optical fiber 108. The light-emitting side optical fiber 106 is connected to the trapezoidal flat plate 102 and the light-emitting apparatus 30. The light-receiving side optical fiber 108 is connected to the trapezoidal flat plate 102 and the light-analyzing apparatus 40.

The light-emitting apparatus 30 emits the testing light 302 entering the trapezoidal flat plate 102 through the light-emitting side optical fiber 106, so that the testing light 302 is refracted and reflected in the trapezoidal flat plate 102 to generate the total reflections and leaves from the trapezoidal flat plate 102 to generate the external refracted light 304. The light-analyzing apparatus 40 receives the external refracted light 304 through the light-receiving side optical fiber 108 to analyze the external refracted light 304.

The light-emitting side optical fiber 106 is, for example but not limited to, a plastic optical fiber (POF). The light-receiving side optical fiber 108 is, for example but not limited to, a plastic optical fiber. A refractive index of the light-emitting side optical fiber 106 is, for example but not limited to, less than a refractive index of the trapezoidal flat plate 102. A refractive index of the light-receiving side optical fiber 108 is, for example but not limited to, less than the refractive index of the trapezoidal flat plate 102. Optical fiber leading-in holes (not shown in FIG. 3) are arranged at two sides of the trapezoidal flat plate 102, so that the optical fibers or the optical fiber collimators (not shown in FIG. 3) are able to directly insert into the optical fiber leading-in holes to achieve the light focusing (or the beam focusing). The present invention further comprises a base (not shown in FIG. 3) rendering that the trapezoidal flat plate 102, the light-emitting side optical fiber 106 and the light-receiving side optical fiber 108 are able to be arranged on the base stably.

In an embodiment of the present invention, an incident angle of the testing light 302 entering the trapezoidal flat plate 102 is, for example but not limited to, eight degrees. When the incident angle of the testing light 302 entering the trapezoidal flat plate 102 is greater than eight degrees, the reflected light belongs to the high order mode in the optical fiber. The feature of the high order mode is that the energy attenuates fast, so that the influence from the reflected light to the original light source can be reduced. The descriptions of the elements shown in FIG. 3 which are the same as the elements shown in FIG. 2 are not repeated here for brevity.

The advantage of the present invention is to arrange the surface plasmon resonance sensing platform easily and increase the signal strength. Moreover, if the trapezoidal flat plate 102 is replaced by a rectangular flat plate, the testing light 302 enters the rectangular flat plate vertically, so that the reflected light follows the original path to be reflected back to the laser light source or the light emitting diode light source, so that the laser light source or the light emitting diode light source is not stable. Moreover, the testing light 302 entering the rectangular flat plate is parallel to the rectangular flat plate, so that the main energy of the testing light 302 entering the rectangular flat plate is not able to touch the upper base of the rectangular flat plate, but only a small amount of the energy is able to touch the upper base of the rectangular flat plate to generate the surface plasmon resonance phenomenon.

The present invention utilizes the trapezoidal flat plate 102. The reflected light which is generated by the (optical fiber) light source touching the glass interface has the double reflected angle, so that the reflected light is not to be reflected back to the (optical fiber) light source. Namely, the reflected light does not impact the (optical fiber) light source. Under the proper design of the trapezoidal flat plate 102, the refracted light which is generated by the (optical fiber) light source entering the glass interface is able to proceed to the upper base 1022 of the trapezoidal flat plate 102. Namely, the main energy is able to touch the upper base 1022 of the trapezoidal flat plate 102 to generate the stronger surface plasmon resonance phenomenon. With a plurality of the total reflections, the signal strength is increased finally. It is noted that even though only two total reflections are shown in FIGS. 1-3, the present invention is not limited to it. Under the proper design of the trapezoidal flat plate 102 (for examples, prolonging the upper base 1022 of the trapezoidal flat plate 102 and the lower base 1024 of the trapezoidal flat plate 102, or changing the tilt angles of the light-entering broadside 1026 of the trapezoidal flat plate 102 and the light-leaving broadside 1028 of the trapezoidal flat plate 102), the present invention is able to comprise more than two total reflections to increase the signal strength.

The present invention utilizes the trapezoidal flat plate 102 as the core (namely, the key element). The present invention has features of the mature coating process, high yield rate, carrying conveniently, low cost and strong structure. The present invention provides a simple and cheap surface plasmon resonance sensing platform, so that the disadvantages of the related art surface plasmon resonance sensing platforms comprising the difficultly processing, high cost, unable to largely produce and too complicated structure can be solved. The present invention can be applied to the drug research, medical diagnosis, environmental monitoring and food safety.

Although the present invention has been described with reference to the preferred embodiment thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

1. A trapezoidal flat plate type surface plasmon resonance sensing platform applied to a sensed object, a light-emitting apparatus and a light-analyzing apparatus, the trapezoidal flat plate type surface plasmon resonance sensing platform comprising:
a trapezoidal flat plate; and
a metal layer arranged on the trapezoidal flat plate,
wherein the trapezoidal flat plate comprises a light-entering lateral side and a light-leaving lateral side;
wherein the sensed object is arranged on the metal layer; the light-emitting apparatus emits a testing light entering the trapezoidal flat plate through the light-entering lateral side, so that the testing light is refracted and reflected in the trapezoidal flat plate to generate a plurality of total reflections and leaves from the trapezoidal flat plate through the light-leaving lateral side to generate an external refracted light; the light-analyzing apparatus receives the external refracted light to analyze the external refracted light;
wherein the total reflections are used to excite a plurality of free electrons of the metal layer to generate a surface plasmon resonance phenomenon.

2. The trapezoidal flat plate type surface plasmon resonance sensing platform of claim 1, wherein the trapezoidal flat plate comprises an upper base and a lower base; a length of the upper base of the trapezoidal flat plate is longer than a length of the lower base of the trapezoidal flat plate; the upper base of the trapezoidal flat plate is parallel to the lower base of the trapezoidal flat plate; the metal layer is arranged on the upper base of the trapezoidal flat plate; the testing light is refracted in the trapezoidal flat plate and is reflected on the lower base of the trapezoidal flat plate to generate the total reflections on the upper base of the trapezoidal flat plate.

3. The trapezoidal flat plate type surface plasmon resonance sensing platform of claim 1, wherein a length of the light-entering lateral side of the trapezoidal flat plate is equal to a length of the light-leaving lateral side of the trapezoidal flat plate; the light-entering lateral side of the trapezoidal flat plate is not parallel to the light-leaving lateral side of the trapezoidal flat plate.

4. The trapezoidal flat plate type surface plasmon resonance sensing platform of claim 1, wherein an incident angle of the testing light entering the trapezoidal flat plate is eight degrees.

5. The trapezoidal flat plate type surface plasmon resonance sensing platform of claim 1, wherein the trapezoidal flat plate and the metal layer are integrated as a removable module.

6. The trapezoidal flat plate type surface plasmon resonance sensing platform of claim 1, wherein the metal layer is sputtered on the trapezoidal flat plate.

7. The trapezoidal flat plate type surface plasmon resonance sensing platform of claim 1, wherein the metal layer is divided into a sensing area array to sense a plurality of the sensed objects.

8. The trapezoidal flat plate type surface plasmon resonance sensing platform of claim 1, wherein the trapezoidal flat plate is a trapezoidal glass flat plate.

9. The trapezoidal flat plate type surface plasmon resonance sensing platform of claim 1, wherein the light-analyzing apparatus is a frequency spectrum analyzer.

10. The trapezoidal flat plate type surface plasmon resonance sensing platform of claim 1, wherein the sensed object is a saliva sample, a blood sample, a hair sample, an egesta sample or a cell sample.
